(19) United States
(12) Patent Application Publication
(10) Pub. No.: US 2022/0244279 A1
(43) Pub. Date: Aug. 4, 2022

(54) ASSAYS AND METHODS FOR SCREENING FOR CARDIOVASCULAR DISEASE USING LDL SUBCLASSES AND ENDOTHELIAL NITRIC OXIDE/PEROXYNITRITE BALANCE

(71) Applicant: Ohio University, Athens, OH (US)

(72) Inventor: Tadeusz Malinski, Athens, OH (US)

(73) Assignee: Ohio University, Athens, OH (US)

(21) Appl. No.: 17/612,851

(22) PCT Filed: May 18, 2020
(86) PCT No.: PCT/US2020/033351

§ 371 Date: Nov. 19, 2021

Related U.S. Application Data
(60) Provisional application No. 62/853,224, filed on May 28, 2019.

(51) Int. Cl. G01N 33/92; G01N 33/84

(52) U.S. Cl. CPC G01N 33/92; G01N 33/84; G01N 2800/52; G01N 2800/32; G01N 2800/50

(57) ABSTRACT

Assays and methods for diagnosing whether a subject has a cardiovascular disease (CVD) by measuring the concentrations of nitric oxide [NO] and peroxynitrite [ONOO⁻] stimulated by the different subclasses of LDL in one or more cells of the subject are described.

### RELATED APPLICATIONS

This application claims priority to U.S. Provisional Application Ser. No. 62/853,224 filed under 35 U.S.C. § 111(b) on May 28, 2019, the disclosure of which is incorporated herein by reference in its entirety.

### STATEMENT REGARDING FEDERALLY SPONSORED RESEARCH

This invention was made with no government support. The government has no rights in this invention.

### BACKGROUND OF THE INVENTION

Low density lipoprotein (LDL) transports fat molecules through bloodstream. Both native-LDL (n-LDL) and oxidized-LDL (ox-LDL) have been considered as bad cholesterol because of an association with several cardiovascular diseases. There are three major subclasses of LDL with distinct densities: n-LDL subclass A contains more of the larger and less dense LDL particles (density of 1.025-1.034 g/mL); an intermediate group, n-LDL subclass I has density of 1.034-1.044 g/mL; and finally, n-LDL subclass B, which has more smaller and denser LDL particles (density of 1.044-1.060 g/mL).

It is well-established that most of the cholesterol in vascular circulation involves LDL. The Framingham Heart Study conclusively shows that the risk of coronary heart disease (CHD) is associated with a high level of cholesterol (mainly LDL). The most common approach to determine LDL is the Friedewald Calculation, based on triglycerides and HDL. This calculation suffers from several limitations. Recently, methods of direct measurements of LDL have been introduced. These methods are call homogeneous methods and include ultracentrifugation, LC/MS, GC electrophoresis, solvent extraction, chemical precipitation, immunoseparation, nuclear magnetic resonance (NMR), magnetic precipitation, as well as other homogeneous enzymatic assays.

These methods of LDL determination are reasonably accurate, especially compared to the Friedewald calculation, and reasonably specific with an accuracy of about 4-5%. Therefore, the general determination of the total LDL level is not a major problem in medical diagnosis. The problem, rather, is in the poor correlation between LDL levels and the development of coronary heart disease.

About 80% of patients (in the USA) brought to the emergency room due to a heart attack have “normal” cholesterol levels, of LDL considered to be “bad cholesterol.” According to clinical studies, this “bad cholesterol” can sometimes be “very bad”, while other times it's not bad at all and can be observed at the normal range LDL plasma concentration of <100 mg/dl.

The current methods for the measurement of total LDL are precise, accurate and reproducible. However, a diagnosis based on these widely-used measurements is not reliable, and is very often misleading. It is the cause of severe diagnostic burden on the health care system and leads to a significant loss of life, especially for the younger generation of patients. Therefore, the measurements of total LDL as a diagnostic tool in medicine should be drastically changed. High LDL does not always provide a true representation of an increase in cardiovascular risk, just as normal or low LDL is not always an objective indicator that this risk is negligible.

Still, the current criteria for diagnosis of the potential damaging effect of LDL to the cardiovascular system is misleading, and are no longer suitable to be used for the early and long-term diagnosis of LDL as a risk factor in the development of cardiovascular diseases (CVD).

There is no admission that the background art disclosed in this section legally constitutes prior art.

### SUMMARY OF THE INVENTION

In a first aspect, there is provided a method of determining potential risk to a subject's cardiovascular system, comprising:

measuring, in situ, the concentrations of nitric oxide [NO] and peroxynitrite [ONOO⁻] stimulated by the different subclasses of LDL in one or more cells of the subject;

wherein the concentration ratio of [NO]/[ONOO⁻], when this concentration ratio falls below 1.0, it indicates an imbalance between cytoprotective NO and cytotoxic ONOO⁻.

In another aspect, there is provided a method for diagnosing general cardiac risk factors based on the measurement of subclass B of LDL, comprising:

measuring the amount/concentration of LDL-B by ultracentrifugation or electrophoresis methods; and,

analyzing algorithm to determine the level of LDL-B compared to the total level of LDL;

wherein a score in % is then compared with a calibration curve, based on nanomedical measurements of NO and ONOO− concentrations, which are produced by a model of endothelial cells (mixed population); and, wherein a score of 40%, or higher, on this scale indicates increased risk of CVD.

In another aspect, there is provided a method for diagnosing cardiovascular risk are based on the simultaneous measurements of all the subclasses of LDL (A, I and B), comprising:

i) separating three subclasses from a sample, and quantitatively measured using either electrophoresis, ultracentrifugation, or immunoseparation methods;

ii) comparing the amount/concentration of each LDL subclass, separately; thereafter,

iii) comparing the amount/concentration of LDL-B to the sum of LDL-I and LDL-A concentrations; and,

iv) constructing a calibration curve from the calibration data from NO and ONOO− concentrations produced by endothelial cells after stimulation with different combinations of LDL-A, LDL-B and LDL-I.

In another aspect, there is provided a method for personalized diagnosis of a patient to estimate the risk for cardiovascular disease (CVD), endothelial dysfunction, and/or the rate and time of the progression of vascular disease comprising:

i) harvesting endothelial cells and/or platelets from the patient;

ii) stimulating NO and ONOO− concentrations generated by the endothelial cells and/or platelets with each subclass of LDL, both separately and in combination; AB, AI and BI by:
a) separating three subclasses from a sample, and quantitatively measured using either electrophoresis, ultracentrifugation, or immunoseparation methods;
b) comparing the amount/concentration of each LDL subclass, separately; thereafter,
c) comparing the amount/concentration of LDL-B to the sum of LDL-I and LDL-A concentrations; and,
d) constructing a calibration curve from the calibration data from NO and ONOO-concentrations produced by endothelial cells after stimulation with different combinations of LDL-A, LDL-B and LDL-I;

iii) determining a personal diagnosis based to accurately estimate the risk for CVD, as well as the level of endothelial dysfunction, the rate and time of the progression of vascular disease; and,

iv) administering a suitable pharmacological treatment, optionally, using selective LDL-B statins, L-arginine, vitamin D3 and others.

In another aspect, there is provided a method of determining potential risk to a subject's cardiovascular system, comprising:

measuring, in situ, the concentrations of nitric oxide [NO] and peroxynitrite [ONOO—] stimulated by the different subclasses of LDL in at least one cell of the subject;

wherein a ratio of [NO]/[ONOO—] is below 1.0 indicates an imbalance between cytoprotective NO and cytotoxic ONOO—.

In another aspect, there is provided a method of determining potential risk to a subject's cardiovascular system, comprising:

i) obtaining a sample from the subject having at least one cell having low density lipoproteins (LDL) therein;

wherein the LDLs are comprised of the subclasses of LDL with distinct densities:
n-LDL subclass A which contains more of the larger and less dense LDL particles (density of 1.025-1.034 g/mL);
an intermediate group, n-LDL subclass I which has density of 1.034-1.044 g/mL; and,
n-LDL subclass B, which has more smaller and denser LDL particles (density of 1.044-1.060 g/mL);

ii) measuring concentration of NO and ONOO⁻ released from the cell employing nanosensors with a diameter of <300 nm; and,

iii) determining the ratio of cytoprotective NO concentration to cytotoxic ONOO⁻ concentration [NO]/[ONOO⁻];

wherein a balance of [NO]/[ONOO⁻] in normal endothelium is about 5, but is shifted to 2.7±0.4, 0.5±0.1 and 0.9±0.1 for n-LDL subclasses A, B and I, respectively; and,

wherein a ratio below 1.0 indicates an imbalance between cytoprotective NO and cytotoxic ONOO⁻, which negatively affects endothelial function.

In certain embodiments, a high content/level of subclass B LDL in total cholesterol is a determinant of potential risk for the subject's cardiovascular system.

In another aspect, there is provided an assay for diagnosing whether a subject has cardiovascular disease (CVD), comprising:

i) obtaining, or having obtained, at least one cell from the subject;

ii) exposing the sample to a quantity of nanosensors in an amount sufficient to measure the concentration of NO and ONOO⁻ released from the cell, and,
iii) determining the ratio of cytoprotective NO concentration to cytotoxic ONOO⁻ concentration [NO]/[ONOO⁻];

wherein a balance of [NO]/[ONOO⁻] in normal endothelium is about 5, but is shifted to 2.7±0.4, 0.5±0.1 and 0.9±0.1 for native LDL (n-LDL) subclasses A, B and I, respectively; and,

wherein a ratio below 1.0 indicates an imbalance between cytoprotective NO and cytotoxic ONOO⁻.

In certain embodiments, a high content/level of subclass B LDL in total cholesterol is a determinant of potential risk for the cardiovascular system.

In certain embodiments, the nanosensors comprise chemically modified carbon-fibers.

In certain embodiments, the nanosensors comprise a NO sensing material and an ONOO⁻ sensing material deposited on the tip of a carbon fiber.

In certain embodiments, the NO sensing material comprises a conductive film of polymeric nickel (II) tetrakis (3-methoxy-4hydroxy-phenyl) porphyrinic; and/or wherein the ONOO⁻ sensing material comprises a polymeric film of Mn (III)paracyclophanyl-porphyrin.

In certain embodiments, NO and ONOO⁻ released are measured by using amperometry with time (detection limit of 1 nmol/L and resolution time <50 ms).

In certain embodiments, the nanosensors are calibrated by using linear calibration curves from 50 nmol/L to 1000 nmol/L and/or standard addition methods before and after measurements with aliquots of NO or ONOO⁻ standard solutions, respectively.

In another aspect, there is provided a method for diagnosing whether a subject has cardiovascular disease (CVD), comprising: determining CVD progression in the subject by measuring the increase of subclass B LDL in a sample from the subject, as compared to subclasses A and I and/or a previous measured sample from the subject.

In certain embodiments, a balance of [NO]/[ONOO⁻] in normal endothelium is about 5, but is shifted to 2.7±0.4, 0.5±0.1 and 0.9±0.1 for native LDL (n-LDL) subclasses A, B and I, respectively; and, wherein a ratio below 1.0 indicates an imbalance between cytoprotective NO and cytotoxic ONOO⁻.

In certain embodiments, the method is used for mass screening of patients.

In certain embodiments, the method further comprises: determining the phase of CVD in the subject by distinguishing among ratios of subclasses A, I and B of LDL.

In another aspect, there is provided a method diagnosing whether a subject has cardiovascular disease (CVD), comprising:

i) obtaining, or having obtained, a sample of at least one cell from a subject;

ii) contacting the sample with a nanosensor; and,

iii) measuring the concentration of NO and ONNO⁻ in the sample, wherein a balance of [NO]/[ONOO⁻] in normal endothelium is about 5, but is shifted to 2.7±0.4, 0.5±0.1 and 0.9±0.1 for native LDL (n-LDL) subclasses A, B and I, respectively; and,

wherein a ratio below 1.0 indicates an imbalance between cytoprotective NO and cytotoxic ONOO⁻.

In another aspect, there is provided a method of treating cardiovascular disease (CVD) in a subject in need thereof, comprising:

i) conducting the assay described herein, and
ii) treating the subject if the assay shows the presence of changes in the ratios of at least one of the subclasses A, I and B of LDL.

In another aspect, there is provided a method of reducing the risk of CVD progression or reducing the risk of recurrence of a CVD in remission in a subject, the method comprising screening for changes in the ratios of at least one of the subclasses A, I and B of LDL.

In another aspect, there is provided an in vitro method for determining a drug-responding or non-responding phenotype in a subject suffering from a CVD, comprising the steps of:

i) determining from a biological sample (from, for example, blood and/or endothelial cells) of said subject the ratios of at least the LDL subclasses (for example, subclass B to A+I or B to A);

ii) comparing the level in step a) to a reference level; and,

iii) determining the drug-responding or non-responding phenotype from said comparison.

In another aspect, there is provided a method for designing or adapting a treatment regimen for a subject suffering from CVD, comprising the steps of:
i) determining from a biological sample of said subject a drug-responding or non-responding phenotype according to the methods described herein; and
ii) designing or adapting a treatment regimen for said subject based upon said responding or non-responding phenotype.

In another aspect, there is provided a method for diagnosing CVD wherein an in-vivo sample is quantified, the method comprising:

i) obtaining, or having obtained, an in-vivo sample from a patient suspected of having CVD;

ii) contacting the patient sample with one or more nanosensors;

iii) detecting and/or quantifying ratios of the subclasses B to A+I, or B to A ratio of LDL present in the patient sample; thereby obtaining a patient sample subclass-LDL value;

iv) comparing the patient sample value to a reference value; wherein the reference value is set based on one or more individuals that do not have CVD; and,

v) diagnosing CVD if the patient sample value is below the reference value.

In certain embodiments, the reference value is set by:

a) obtaining one or more in-vivo normal samples from one or more individuals that do not have CVD;

b) contacting the one or more normal samples with a nanosensor;

c) detecting and/or quantifying ratios of at least one of the subclasses A, I and B of LDL present in the one or more normal samples; thereby obtaining one or more reference values; and,

d) setting the reference value based on the one or more normal sample values.

In certain embodiments, the method further comprises:

e) applying the measured value from the subject against a database of measured values from control subjects, wherein the database is stored on a computer system; and,

f) determining that the subject has an increased risk of having CVD or disorder by measuring a change of at least 5% in the value relative to measured value from control subjects.

In certain embodiments, a subject has a risk of having or has CVD if the measurements show a change in expression of at 10% compared to a control subject population.

In certain embodiments, the subject who has a change in expression of at least 10% compared to a control subject population, is further screened for CVD.

In another aspect, there is provided a computer-based method for screening a subject for the presence of and treating CVD, comprising: screening the subject by:

i) obtaining, or having obtained, a sample comprising at least one cell from the subject;

ii) exposing the sample to a quantity of nanosensors in an amount sufficient to measure the concentration of NO and ONOO⁻ released from the cell;

iii) determining the ratio of cytoprotective NO concentration to cytotoxic ONOO⁻ concentration [NO]/[ONOO⁻];
wherein a balance of [NO]/[ONOO⁻] in normal endothelium is about 5, but is shifted to 2.7±0.4, 0.5±0.1 and 0.9±0.1 for native LDL (n-LDL) subclasses A, B and I, respectively; and,
wherein a ratio below 1.0 indicates an imbalance between cytoprotective NO and cytotoxic ONOO⁻;

iv) applying a measured ratio value from the subject against a database of measured ratio values from control subjects, wherein the database is stored on a computer system;

v) determining that the subject has an increased risk of having CVD by measuring a change of at least 5% in the subject's ratio value relative to measured ratio values from control subjects;

vi) performing a diagnostic procedure comprising downloading the plurality of subject's ratio values into a computer processor; and,

vii) administering an effective anti-CVD treatment to the subject.

In certain embodiments, the anti-CVD treatment comprises administering an effective amount of a composition to restore the catalytic function of NOS in the subject's cells and/or the decrease of subclass B LDL concentration.

In certain embodiments, the pharmaceutical compositions are selected from one or more:

combinations of vitamin D₃, L-arginine, apocynin, statins with selected capability to restore damage imposed by subclass B of LDL on NOS function.

In another aspect, there is provided a kit intended for the detection of CVD in a sample, the kit comprising: a quantity of nanosensors, and instructions for obtaining a ratio value of cytoprotective NO concentration to cytotoxic ONOO⁻ concentration [NO]/[ONOO⁻].

In certain embodiments, a balance of [NO]/[ONOO—] in normal endothelium is about 5, but is shifted to 2.7±0.4, 0.5±0.1 and 0.9±0.1 for n-LDL subclasses A, B and I, respectively; and, wherein a ratio below 1.0 indicates an imbalance between cytoprotective NO and cytotoxic ONOO—, which affects endothelial function.

In certain embodiments, the nanosensor comprises a chemically modified carbon-fibers.

In certain embodiments, the nanosensor comprises a NO sensing material and an ONOO— sensing material deposited on the tip of a carbon fiber.

In certain embodiments, the NO sensing material comprises a conductive film of polymeric nickel (II) tetrakis (3-methoxy-4hydroxy-phenyl) porphyrinic; and/or wherein the ONOO— sensing material comprises a polymeric film of Mn (III)paracyclophanyl-porphyrin.

In certain embodiments, NO and ONOO— released are measured by using amperometry with time (detection limit of 1 nmol/L and resolution time <50 ms).

In certain embodiments, the nanosensor is calibrated by using linear calibration curves from 50 nmol/L to 1000 nmol/L and/or standard addition methods before and after measurements with aliquots of NO or ONOO— standard solutions, respectively.

Various objects and advantages of this invention will become apparent to those skilled in the art from the following detailed description of the preferred embodiment, when read in light of the accompanying drawings.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIGS. 1A-1B: Amperograms (current calibrated as concentration vs time) of NO and ONOO⁻ release stimulated by LDL with different patterns on the surface of endothelial cells:

FIG. 1A) NO release from endothelial cells stimulated by LDL (Pattern A, B and 1, 1000 μg/mL).

FIG. 1B) ONOO⁻ release from endothelial cells stimulated by LDL (Pattern A, B and I, 1000 μg/mL). Arrows indicate LDL injection.

FIGS. 2A-2B: Maximal [NO] and [ONOO⁻] release from the surface of endothelial cells stimulated by LDL with different patterns:

FIG. 2A) Maximal [NO] and [ONOO⁻] release from endothelial cells stimulated by LDL (Pattern A, B and I, 1000 μg/mL), solid bar indicates [NO] and open bar indicates [ONOO⁻].

FIG. 2B) A ratio of maximal [NO] to [ONOO⁻]. Data are expressed as mean±SD. Significance was determined using Student's t-test. *P<0.01 vs B.

FIGS. 3A-3C: Dose-dependent NO and ONOO⁻ release from the surface of endothelial cells stimulated by LDL:

FIG. 3A) Production of NO stimulated by LDL with different patterns (A, B and I) and different concentrations (from 50 μg/mL to 1000 μg/mL).

FIG. 3B) Production of ONOO⁻ stimulated by LDL with different patterns (A, B and I) and different concentrations (from 50 μg/mL to 1000 μg/mL).

FIG. 3C) The ratio of [NO] to [ONOO⁻]. Black triangle, white circle and black dot indicate LDL injection of pattern A, B and I, respectively.

FIGS. 4A-4B: [NO] and [ONOO⁻] release stimulated by LDL mixture with different combinations:

FIG. 4A) [NO] and [ONOO⁻] production stimulated by LDL mixture (800 μg/mL). Solid bar indicates [NO] and open bar indicates [ONOO⁻].

FIG. 4B) Ratio of [NO] to [ONOO⁻]. Data are expressed as mean±SD.

FIGS. 5A-5C: [NO] and [ONOO⁻] release from endothelial cells stimulated by LDL after incubation with different treatments. Endothelial cells were incubated with control (endothelial basal medium EBM), PEG-SOD (400 U/mL), L-arginine (300 μM), sepiapterin (200 μM), Vascular cell adhesion molecule-1 (L-NAME) (100 μM) and VAS2870 (10 μM) at 37° C. for 30 minutes:

FIG. 5A) NO production stimulated by LDL (Pattern A, B and I, 800 μg/mL).

FIG. 5B) ONOO⁻ production stimulated by LDL (Pattern A, B and I, 800 μg/mL).

FIG. 5C) Ratio of [NO] to [ONOO⁻]. Data are expressed as mean±SD.

FIGS. 6A-6C: [NO] and [ONOO⁻] release stimulated by ox-LDL/n-LDL:

FIG. 6A) NO production stimulated by ox-LDL/n-LDL (800 μg/mL).

FIG. 6B) ONOO⁻ production stimulated by ox-LDL/n-LDL (800 μg/mL).

FIG. 6C) Ratio of [NO] to [ONOO⁻]. Data are expressed as mean±SD. Significance was determined using Student's t-test. *P<0.01 vs n-LDL.

FIGS. 7A-7C: Monocyte adhesion and cell adhesion molecular expression stimulated by pattern A, B and I LDL:

FIG. 7A) Monocyte adhesion stimulated by pattern A, B and I LDL (400 μg/mL) measured at different incubation time (from 10 minutes to 60 minutes).

FIG. 7B) Dose-dependent monocyte adhesion stimulated by pattern A, B and I LDL (50, 100, 200, 400 μg/mL). Data are expressed as mean±SD. MFI indicates mean fluorescence intensity.

FIG. 7C) Effect of LDL with different patterns on the expression of Intercellular adhesion molecule-1 (ICAM-1) and Vascular cell adhesion molecule-1 (VCAM-1). Endothelial cells were incubated with LDL of pattern A, B and I (400 μg/mL) at 37° C. for 5 hours. After incubation, the cells were washed with DPBS and fixed with 4% formaldehyde solution. ICAM-1 and VCAM-1 expression were determined by cell ELISA. Data are expressed as mean±SD. Solid bar indicates ICAM-1, open bar indicates VCAM-1. OD indicates optical density. Significance was determined using Student's t-test. *P<0.01 vs control.

FIGS. 8A-8C: Monocyte adhesion and cell adhesion molecular expression stimulated by n-LDL/ox-LDL of pattern A, B and I:

FIG. 8A) Monocyte adhesion stimulated by n-LDL/ox-LDL of pattern A, B and I (400 μg/mL). Data are expressed as mean±SD. MFI indicates mean fluorescence intensity.

FIG. 8B) Effect of n-LDL/ox-LDL with different patterns on the expression of ICAM-1. Endothelial cells were incubated with n-LDL/ox-LDL of pattern A, B and I (400 μg/mL) at 37° C. for 5 hours. After incubation, the cells were washed with DPBS and fixed with 4% formaldehyde solution. ICAM-1 and VCAM-1 expression were determined by cell ELISA.

FIG. 8C) Effect of n-LDL/ox-LDL with different patterns on the expression of VCAM-1. Data are expressed as mean±SD. OD₄₅₀ indicates optical density. Significance was determined using Student's t-test. *P<0.01 vs n-LDL.

### DETAILED DESCRIPTION OF THE INVENTION

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below. Additionally, unless otherwise specifically noted, articles depicted in the drawings are not necessarily drawn to scale.

Throughout this disclosure, various publications, patents, and published patent specifications are/may be referenced by an identifying citation. Such disclosures of these publications, patents, and published patent specifications are hereby incorporated by reference into the present disclosure in their entirety to more fully describe the state of the art to which this invention pertains.

### Definitions

In order to facilitate review of the various embodiments of the disclosure, the following explanations of specific terms are provided:

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like.

As used herein, “therapeutic” is a generic term that includes both diagnosis and treatment. It will be appreciated that in these methods the “therapy” may be any therapy for treating a disease including, but not limited to, pharmaceutical compositions, gene therapy and biologic therapy such as the administering of antibodies and chemokines. Thus, the methods described herein may be used to evaluate a patient or subject before, during and after therapy, for example, to evaluate the reduction in disease state.

As used herein, “adjunctive therapy” is a treatment used in combination with a primary treatment to improve the effects of the primary treatment.

As used herein, “clinical outcome” refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

As used herein, “decrease in survival” refers to a decrease in the length of time before death of a patient, or an increase in the risk of death for the patient.

As used herein, “preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

As used herein, “poor prognosis” generally refers to a decrease in survival, or in other words, an increase in risk of death or a decrease in the time until death. Poor prognosis can also refer to an increase in severity of the disease.

As used herein, “screening” refers to the process used to evaluate and identify candidate patients that are affected by such disease/s.

As used herein, “diagnosing” refers to classifying a medical condition, predicting or prognosticating whether a particular abnormal condition will likely occur or will recur after treatment based on an indicia, detecting the occurrence of the disease in an individual, determining severity of such a disease, and monitoring disease progression.

As used herein, “patient” includes human and non-human animals. The preferred patient for treatment is a human. “Patient,” “individual” and “subject” are used interchangeably herein.

As used herein, “patient” means any individual diagnosed or previously diagnosed as having a disease. This includes individuals previously treated, and displaying remission.

As used herein, “comprising, comprises and comprised of” are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

As used herein, “about” generally refers to a measurable value such as a parameter, an amount, a temporal duration, and the like, is meant to encompass variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

### General Description

Described herein is a nanomedical monitoring system that is to elucidate the molecular mechanism leading to LDL-induced dysfunction of human umbilical-vein endothelial cells (HUVECs). HUVECs form a monolayer on the inner walls of the vasculature. The large surface areas of the vasculature (about 100 m²) is covered by about 0.7-0.8 kg of endothelial cells. Therefore, endothelium can be considered as a major organ in the human body. Dysfunction of this organ can lead to a dysfunction/decrease of efficiency in the blood transport in the vasculature, leading to the development of CHD, heart failure and heart stoppage.

On the molecular level, each of the LDL subclasses interact differently with endothelial cells, stimulating cytoprotective cellular messenger, nitric oxide (NO) and cytotoxic messenger peroxynitrite (ONOO⁻). These molecules were measured simultaneously with nanosensors, and their relative concentration were correlated with the dysfunction of endothelial cells. It is now shown herein that subclass A has a negligible negative influence on endothelial function. Subclass B has a devastating effect on endothelium, while subclass I has an intermediate effect.

It is also shown herein that it is not the total value of LDL, but rather, the relative distribution of LDL subclasses A, I and B that is the most accurate factor in correctly diagnosing potential endothelial dysfunction. Such relative distribution is a useful method, especially for the early diagnosis of potential LDL-induced damage to the cardiovascular system.

With this early and personalized diagnosis of the noxious effects of LDL, there is now the ability to design customized treatments and implement them earlier in the vital process, to reduce the effect of LDL on the development of CHD.

Also described herein are useful assays to test for the early diagnosis of cardiovascular risk associated with LDL. These assays are based on the relative concentration of the subclasses (A, I and B) of LDL and their interaction with endothelial cells, instead of the total level of LDL.

Diagnosis of General Cardiac Risk Factors Based on the Measurement of Subclass B of LDL.

One assay precisely measures the amount/concentration of LDL-B by ultracentrifugation, GC-MS, NMR or electrophoresis methods, and an algorithm to determine the level of LDL-B compared to the total level of LDL. A score in % is then compared with a calibration curve, based on nanomedical measurements of NO and ONOO⁻ concentrations, which are produced by a model of endothelial cells (mixed population). A score of 40%, or higher, on this scale indicates increased risk of CVD. This accuracy of this method is about ±4%.

LDL Diagnostics

Diagnostic methods of cardiovascular risk are based on the simultaneous measurements of all the subclasses of LDL (A, I and B). These three subclasses are separated and quantitatively measured using either electrophoresis, ultracentrifugation, immunoseparation, or GC-MS or NMR methods. A special algorithm compares the amount/concentration of each LDL subclass, separately. Then, the amount/concentration of LDL-B is compared to the sum of LDL-I and LDL-A concentrations. A calibration curve is constructed from the calibration data from NO and ONOO⁻ concentrations produced by endothelial cells after stimulation with different combinations of LDL-A, LDL-B and LDL-I. This provides a high accuracy test with a margin for error of <±3%.

LDL Measuring

Endothelial cells and/or platelets that are harvested/obtained from the diagnosed patients are used for personalized diagnosis and personalized therapies. NO and ONOO⁻ concentrations generated by endothelial cells and/or platelets are stimulated with each subclass of LDL, both separately and in combination; AB, AI and BI. The results from these measurements are included in a specially developed algorithm to produce highly accurate (±2%) results. Based on these results, a personal diagnosis can be applied to accurately estimate the risk for CVD, as well as the level of endothelial dysfunction, the rate and time of the progression of vascular disease, in order to design pharmacological treatments using selective LDL-B statins, L-arginine, vitamin D₃ and others.

### DETAILED DESCRIPTION

n-LDL Subclasses Stimulated NO and ONOO⁻ Release in Endothelial Cells

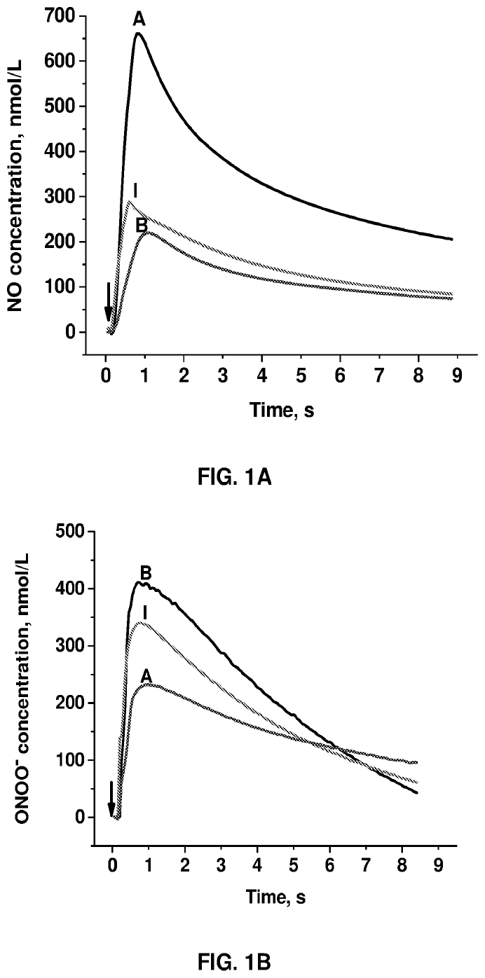

To determine the distinct effect of different subclasses of n-LDL on NO and ONOO⁻ release from human umbilical vein endothelial cells (HUVECs), the real-time production of NO and ONOO⁻ from endothelial cells was measured with nanosensors. A rapid release of NO/ONOO⁻ was detected within 0.1 s after injection of n-LDL, and the maximal concentration of NO and ONOO⁻ were reached within 1.0 s (FIG. 1A, FIG. 1B).

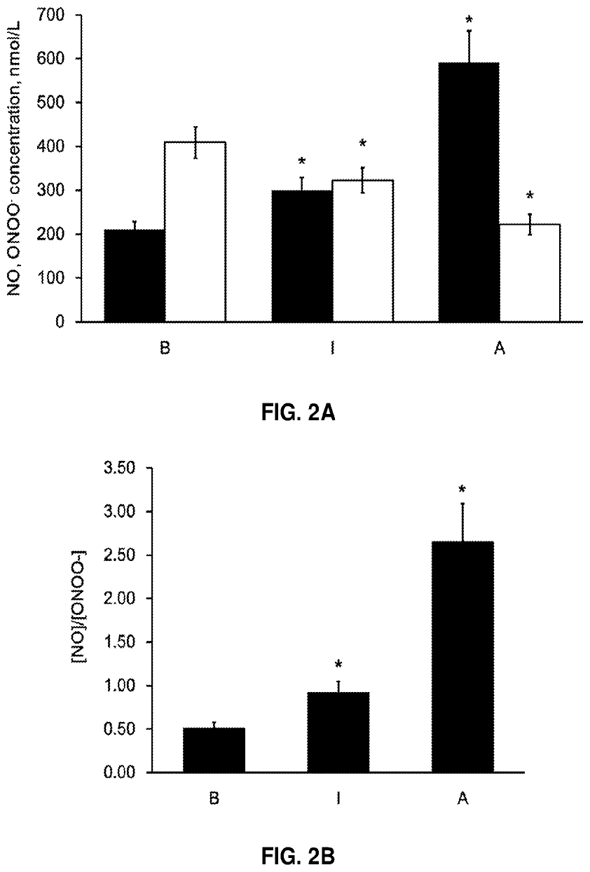

The maximal concentrations of NO and ONOO⁻ released from endothelial cells varied significantly among LDL subclasses A, B and I. Subclass A contains particles with larger size and is less dense than subclass B; and produced the highest concentration of NO. Subclass B consists mainly of n-LDL particles with smaller size and higher density and stimulated the lowest concentration of NO. NO release stimulated by the injection of subclass I is between subclasses A and B. In contrast to NO production, subclass B stimulated the highest level of ONOO⁻, while subclass A produced the lowest level of ONOO⁻ (FIG. 2A).

The ratio of NO concentration, [NO] to the concentration of peroxynitrite, [ONOO⁻] was used to reflect the balance/imbalance between cytoprotective NO and cytotoxic ONOO⁻. High [NO]/[ONOO⁻] ratio indicates a high level of bioavailable, diffusible NO and/or low level of cytotoxic ONOO⁻ (FIG. 2B).

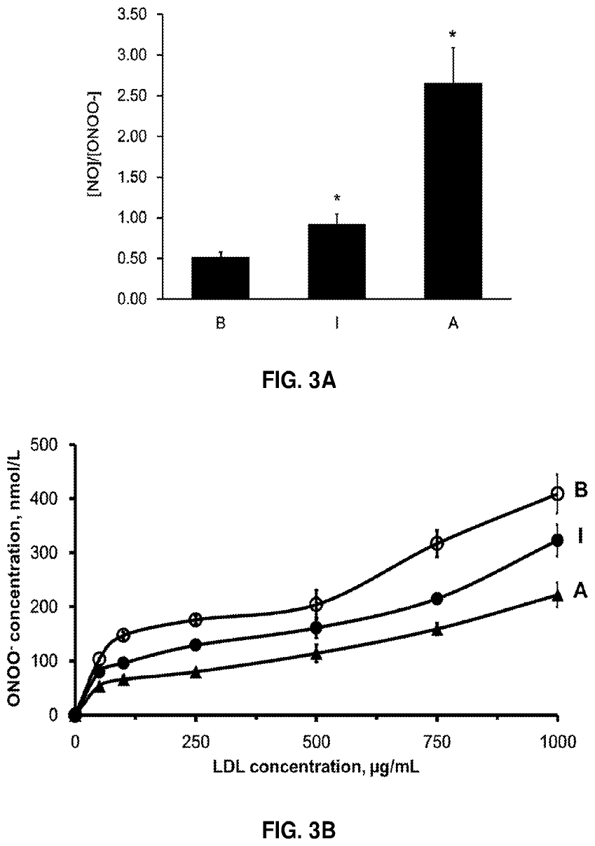

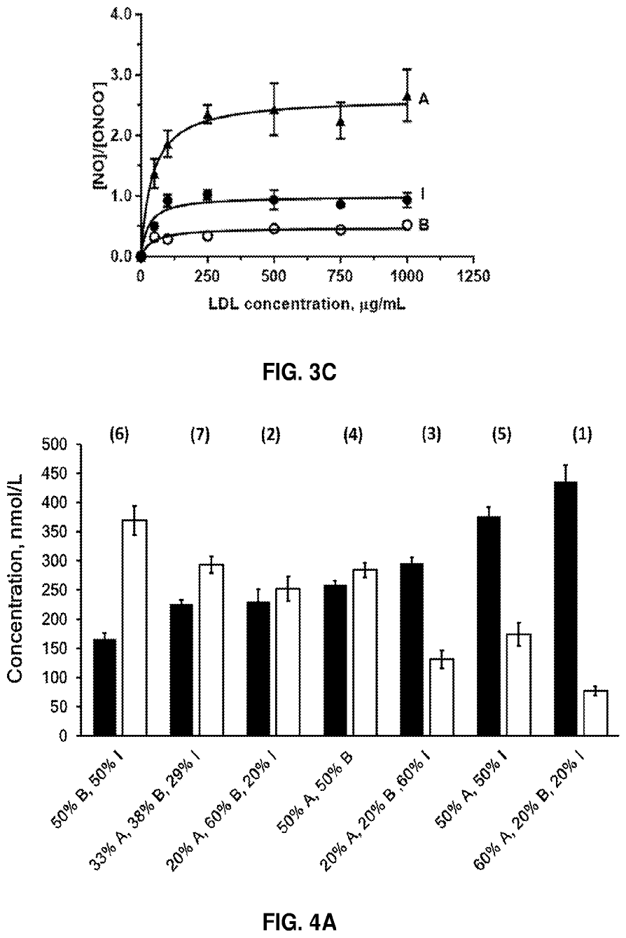

Maximal [NO] and [ONOO⁻] is dose-dependent (FIG. 3A and FIG. 3B). The ratio of [NO]/[ONOO⁻] maintained in the range of about 0.29 to 0.52 for subclass B. For subclasses I and A, the ratios increased variably, with increased LDL, between 0.50 to 0.93 and 1.37 to 2.66, respectively (FIG. 3C).

Effects of the Combinations of Different n-LDL Subclasses on NO and ONOO⁻ Release

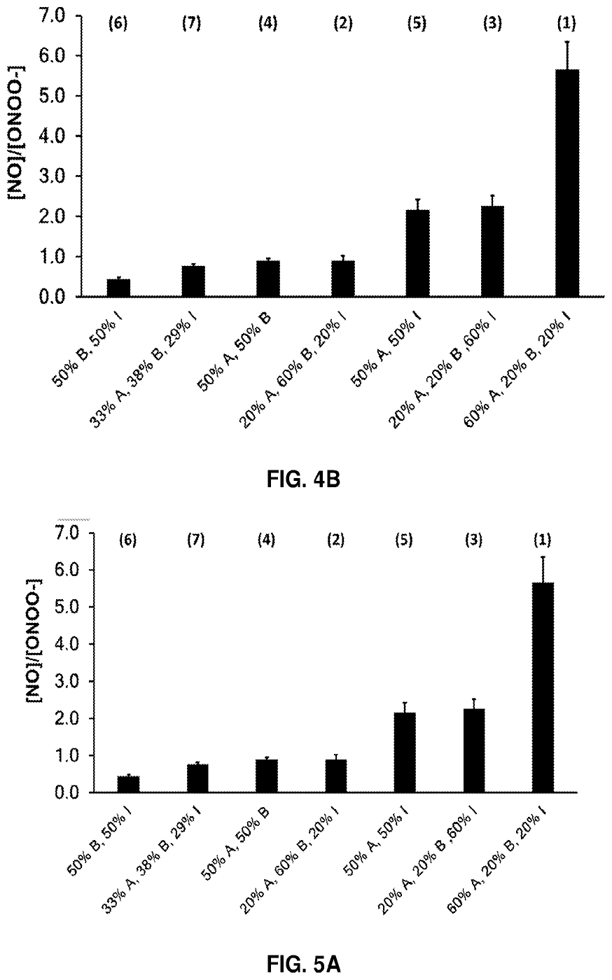

Cells were stimulated with different combination of n-LDL subclass, seven combinations were studied: (1) 60% A, 20% B and 20% I; (2) 20% A, 60% B and 20% I; (3) 20% A, 20% B and 60% I; (4) 50% A and 50% B; (5) 50% A and 50% I; (6) 50% B and 50% I; (7) 33% A, 38% B and 29%. The data showed that group 1(60% A, 20% B and 20% I) produced the lowest concentrations of ONOO⁻ (77±8 nmol/L) and highest concentration of NO (436±28 nmol/L), while group 6 (50% B and 50% I) generated the highest level of ONOO⁻ (369±25 nmol/L) and lowest level of NO (166±10 nmol/L). The ratio of [NO] to [ONOO⁻] concentration was about 5.5 for (1), and about 0.45 for (6) (FIG. 4).

Effect of Modulation in eNOS Pathway on n-LDL Stimulates NO and ONOO⁻ Release

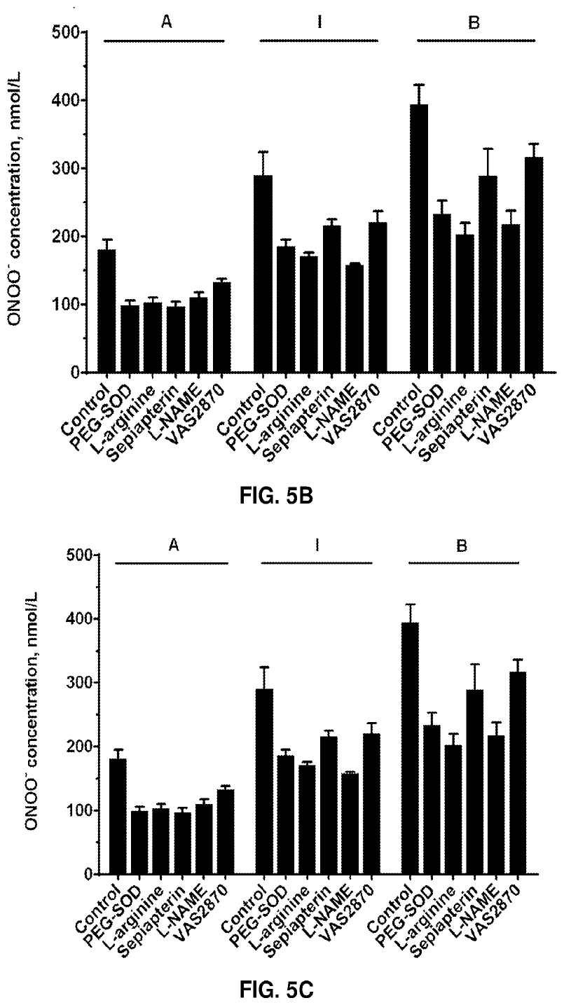

In order to elucidate the kinetics and dynamics of LDL stimulated of NO and ONOO⁻ production, different modulators of endothelial nitric oxide synthase (eNOS) were used. All reagents except L-NAME (eNOS inhibitor) increased NO production after injection of subclasses A, B or I (FIG. 5A).

ONOO⁻ production diminished in the presence of PEG-SOD, L-arginine, sepiapterin, L-NAME and VAS2870 in all standard subclasses (FIG. 5B). With subclass A, the [NO]/[ONOO⁻] ratio remained above one for all of treatments. The ratio for subclass I was greater than one for treatments with L-arginine, sepiapterin and VAS2870, but lower than one for PEG-SOD and L-NAME treatment group. Subclass B revealed a ratio that was below one for all of treatments except for L-arginine (FIG. 5C).

Differences Between n-LDL and Ox-LDL Stimulated NO and ONOO⁻ Release in Endothelial Cells

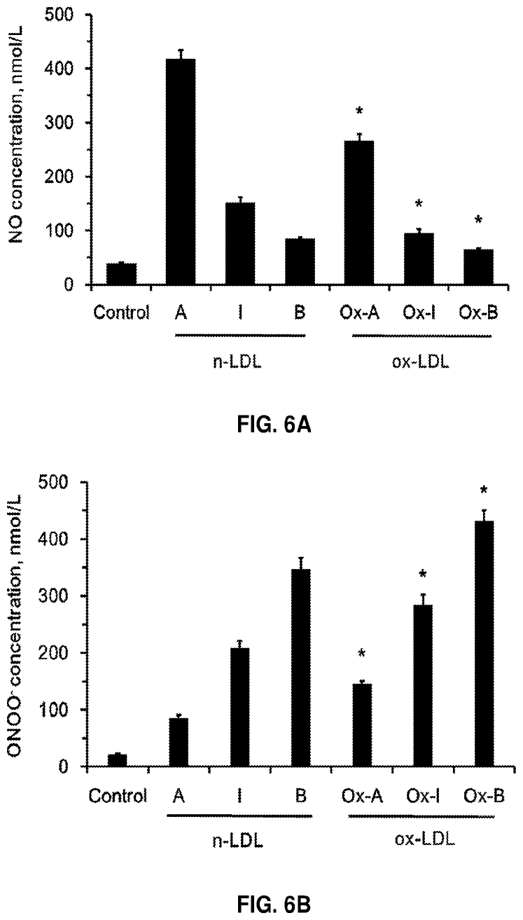

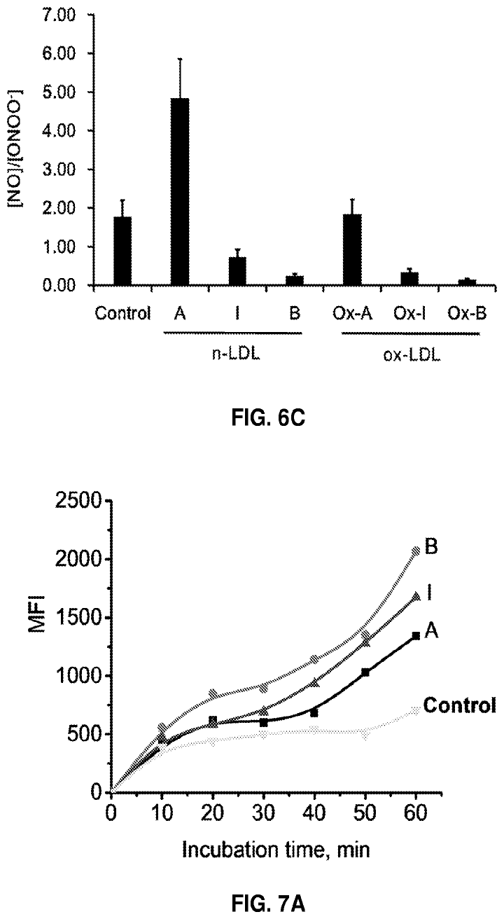

The effects of different subclasses of n-LDL were compared with those of oxidized LDL (ox-LDL). Ox-LDL stimulated NO release at a much lower level than n-LDL, 267±11 vs 418±16 nmol/L for subclass A, 95±7 vs 152±10 nmol/L for subclass I, and 65±3 vs 85±3 nmol/L for subclass B (FIG. 6A).

However, ox-LDL stimulated much higher levels of ONOO⁻ production than n-LDL, 145±6 vs 86±5 nmol/L for subclass A, 284±18 vs 208±13 nmol/L for subclass I, and 432±18 vs 347±20 nmol/L for subclass B (FIG. 6B).

Therefore, [NO] to [ONOO⁻] is 1.84 vs 4.86, 0.33 vs 0.73 and 0.15 vs 0.24 (ox-LDL vs n-LDL) for subclasses A, B and I, respectively (FIG. 6C).

n-LDL-Stimulated Cell Adhesion in Endothelial Cells

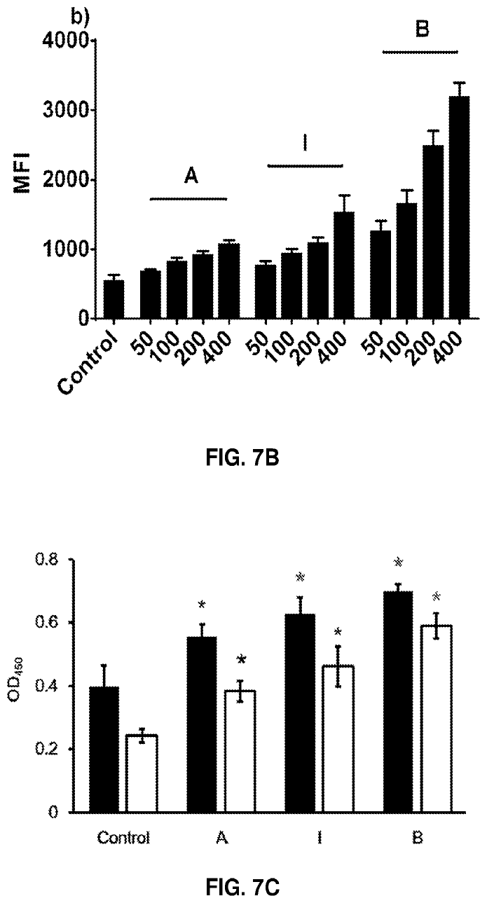

To investigate the effect of different subclasses of n-LDL on monocytes adhesion to endothelial cells, fluorescently pre-labeled THP-1 cells were used. Data showed that the adhesion of monocytes to endothelial cells increased significantly. For subclasses I and A monocytes adhesion was similar, but less extensive than that observed for subclass B (FIG. 7A).

This adhesion increased with time, and after 60 minutes, the differences of THP-1 cells adhesion among treatments with all subclasses (A, B and I) was most significant. The result shows that THP-1 cells adhesion is dose-dependent, 400 μg/mL LDL treatment stimulated the maximal monocytes adhesion while 50 μg/mL LDL treatment stimulated the minimal adhesion. At the same concentration level, n-LDL of different subclasses stimulated monocytes adhesion differently, subclass B stimulated cell adhesion with highest mean-fluorescence-intensity (MFI) while subclass A with lowest MFI (FIG. 7B).

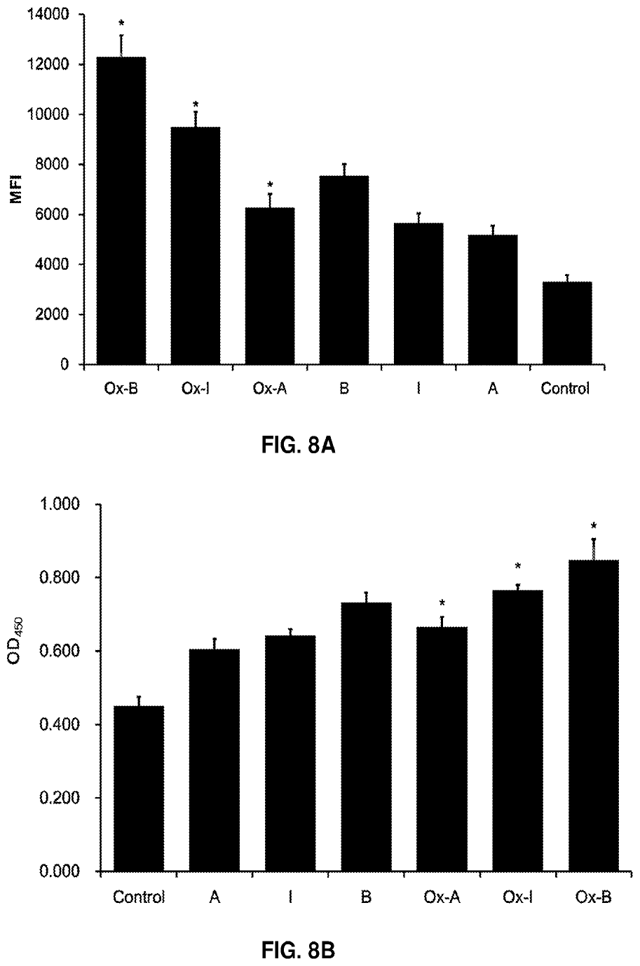

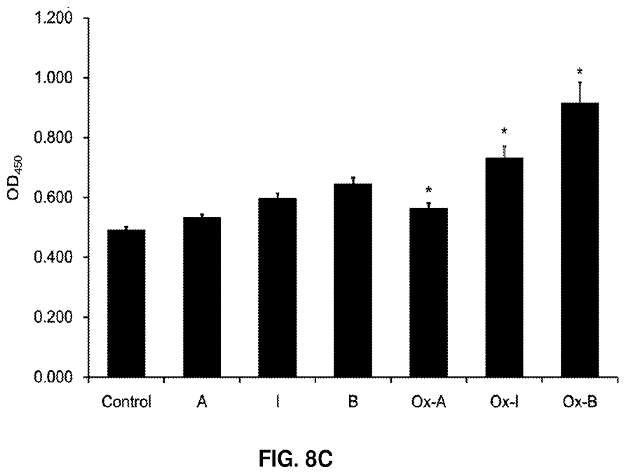

Ox-LDL treatment group stimulated more monocytes adhesion than the n-LDL group, ox-LDL with subclasses A, I and B increased 21%, 73% and 63% of monocytes adhesion than n-LDL with subclasses A, I and B, respectively. Among different subclasses, ox-LDL showed similar results with n-LDL, subclass B stimulated the highest level of cell adhesion (4-fold increase from control), while subclass A stimulated lowest level of cell adhesion (2-fold increase from control), and in between, subclass I stimulated cell adhesion about 3-fold from control (FIG. 8A).

Effects of LDL on ICAM-1 and VCAM-1 Expression in HUVECs

To determine the effect of LDL with different subclasses on ICAM-1 and VCAM-1 expression, endothelial cells were incubated with basal medium containing 400 μg/mL n-LDL or ox-LDL (subclasses A, B and I) for 5 hours and the expression of ICAM-1 and VCAM-1 was measured by cell ELISA. Compared with control, ICAM-1 expression significantly increased to 155±11%, 174±14% and 190±7% of control for LDL of subclasses A, I and B, respectively. VCAM-1 expression also increased in the presence of LDL subclasses A, I and B, similar to the stimulation observed by ICAM-1 (FIG. 7C).

Ox-LDL subclasses increased ICAM-1 expression nearly 20% higher than that observed in n-LDL (FIG. 8B).

Additionally, VCAM-1 expression stimulated by ox-LDL was about 120%, 150% and 190% of control for subclasses A, I and B, which showed 6%, 23% and 42% higher than corresponding n-LDL subclasses, respectively (FIG. 8C).

Discussion

These data show a distinct difference between three major subclasses of n-LDL and ox-LDL in the process of their interactions with endothelium. The nanomedical approach employed here shows, in situ, that after colliding with the membrane of endothelial cells, subclasses A, B and I of LDL can stimulate the production of two signaling molecules: cytoprotective NO and cytotoxic ONOO⁻. The maximal concentrations of NO and ONOO⁻ released differs significantly between each of the subclasses and the relative content of each subclass.

The present method successfully uses the ratio of [NO]/[ONOO⁻] for the precise measurement of eNOS uncoupling, endothelial dysfunction and nitroxidative stress levels (ONOO⁻ vs. protective NO). This nanoanalytical method allows for the simultaneous measurements, in nmol/L, of both NO and ONOO⁻ at near real time (several microseconds) in the femtoliter volume (about 10⁻¹⁵ L) at a constant distance of 5±2 μm from the surface of endothelial cells. This method of simultaneous measurement of NO and ONOO⁻ is used to produce a ratio of the [NO]/[ONOO⁻]. This ratio is useful as a marker of a balance/imbalance between those two molecules, dysfunction of endothelium and level of high oxidative stress. The production of NO by eNOS is always accompanied by the generation of ONOO⁻, which is the product of the reaction between superoxide (O₂⁻) and NO.

This rapid (6×10⁹), diffusion controlled, reaction between NO and O₂⁻ in the biological system prevents the overproduction of NO and/or O₂⁻. In normal, functional endothelium, maximal concentration of ONOO⁻ is about 4-6 times lower than the maximal concentration of NO. The half-life of ONOO⁻ in the biological milieu is less than one second, much shorter than the half-life of NO (about 3-4 s). At low concentrations, ONOO⁻ molecules cannot diffuse any significant distance and are rapidly converted to nontoxic NO₃⁻. At high [NO]/[ONOO⁻] ratio in a normal endothelium, NO signaling, as well as anti-adhesion properties are efficient and the potential for cellular damage by ONOO⁻ (nitroxidative stress) is negligible. However, at high concentrations, the oxidative effect of ONOO⁻ can be severe, especially at low level of cytoprotective NO. At these high concentrations, ONOO⁻ can be protonated and can diffuse, collide with biological molecules and isomerize to initiate a cascade of highly oxidative species—causing oxidative damage to cells, enzymes and DNA leading to endothelial dysfunction, as well as hindered NO signaling and diminished anti-adhesive properties.

With extensive NO production, stimulated by subclass B of LDL, the dimeric form of eNOS became uncoupled and can produce concomitantly NO and O₂⁻, becoming efficient generator of ONOO⁻. With an increase in eNOS uncoupling and endothelial dysfunction, the efficiency of NO signaling decreases exponentially, while the nitroxidative damage to the endothelium increases significantly.

It is now shown herein that with a [NO]/[ONOO⁻] ratio below 1, the peroxynitrite starts to control the redox environment and the protective anti-adhesion role and signaling of NO are greatly diminished.

Using this particular criterion, there is a very significant distinction between the molecular effects of subclasses A, B and I of LDL with their interaction with the endothelium. Subclass A produces a very mild effect in its interaction with endothelium, stimulating NO at moderate levels. A small increase in the level of ONOO⁻ by subclass A indicates the coupling status of eNOS and the efficiency in generating superoxide is minimal. The [NO]/[ONOO⁻] balance is shifted slightly to 2.66±0.43 for subclass A. There is a further decrease in the [NO]/[ONOO⁻] ratio, to 0.93±0.12 for subclass I. But, there is a border line between functional and dysfunctional endothelium, because [NO]/[ONOO⁻] is about one.

Contrary to subclasses A, subclass I, and especially subclass B, have a very significant effect on [NO]/[ONOO⁻] ratio and imbalance between these two molecules. First, subclass B decreases the [NO]/[ONOO⁻] ratio well below 1.0. Under these conditions, the ONOO⁻ becomes a dominating factor in controlling a cytotoxic redox environment in and around endothelial cells. Also, low production of bioavailable NO hinders the rate of diffusion, decreasing the distance and speed of NO signaling. The diminished role of NO in the dysfunction of eNOS is accompanied by an exponential increase in nitroxidative stress imposed by ONOO⁻.

The net result of action of LDL subclass B on endothelium is to decrease both NO signaling, and smooth muscle relaxation as well as, the adhesion of LDL, platelets and leukocytes to the endothelium—all promoted by high levels of ONOO⁻.

Demonstrated herein are contrasting levels of the [NO]/[ONOO⁻] ratio between subclasses A, B and I of LDL cholesterol. Among these three major subclasses, it was found that B imposes the most severe effect on eNOS and endothelial function and subclass I has a more moderate effect on endothelial function. The net effect of a mixture of LDL subclasses A, B and I has on the endothelium is additive, and depends on the content of each of the subclasses. The data also show the subclass-specific differences in both n-LDL and ox-LDL. Subclass B of ox-LDL produced the lowest ratio of [NO]/[ONOO⁻]— about 20% lower than that observed with n-LDL and the lowest among all of the subclasses. In comparison to n-LDL, the effect of decreasing the [NO]/[ONOO⁻] was observed for all ox-LDL subclasses.

Elevated level of LDL may correlate with increased cardiovascular risk. Small and dense subclass of LDL is a key factor that is in strong association with the development of atherosclerosis and other cardiovascular disease (CVD) events. Thus, understanding the role of distinct subclasses of LDL in triggering endothelial dysfunction, as well as the progress of atherosclerosis, facilitates improving accuracy of diagnosis for the evaluation of CVD risk rate.

Also described herein is the role of LDL with different subclasses in induction of NO and ONOO⁻ imbalance in endothelial cells. In these data, the densities of subclasses A, B and I were a little lower; now believed to be due to the ios-osmotic iodixanol gradients that were used for separation of LDL subclasses. Protein molecules of LDL will keep water inside to maintain their native hydrate status, rather than loss of water in highly hyper-osmotic salt gradients, which results in increasing density. The present data show subclass-specific differences in both of n-LDL and ox-LDL stimulated NO and ONOO⁻ release from endothelial cells.

These data show that subclass B can stimulate endothelial cells to produce the highest level of ONOO⁻ and the lowest level of NO, resulting in an imbalance of the [NO]/[ONOO⁻] ratio, which can lead to endothelial dysfunction and aggravate the oxidative stress in endothelial cells.

On the contrary, subclass A stimulated the lowest level of ONOO⁻ and the highest level of NO, keeping the ratio of [NO]/[ONOO⁻] in balance, thus maintaining the functionality of endothelium.

Further, to determine the effect of LDL with different constituents on stimulating NO and ONOO⁻ release from endothelial cells, different mixture combinations of LDL with subclasses A, B and I were evaluated. The most severe combination of LDL consisted of 50% B and 50% I.

It is now believed that the constituents of LDL mixture containing all of three subclasses is related with the release of NO and ONOO⁻. A high percentage of subclass B stimulated a high level of ONOO⁻ and a low level of NO; while high percentage of subclass A stimulated more NO production than ONOO⁻.

Therefore, analyzing the constituents of LDL with different subclasses provides a method for an early medical diagnosis of estimating the risk of cardiovascular disease.

Reagents which can modulate L-arginine/NO pathway, such as PEG-SOD, L-arginine, and sepiapterin, were used to boost the level of bioavailable NO and simultaneously limit the concentration of ONOO⁻, thus favorably increasing the ratio of [NO]/[ONOO⁻].

NO is biosynthesized from L-arginine by eNOS, and thereby as the substrate for NO production, increasing the supplementation of L-arginine and its gradient of concentration can restore the normal status of eNOS and balance of the [NO]/[ONOO⁻] ratio by enhancing NO production. L-arginine treatment with endothelial cells before LDL incubation can increase NO production and decrease ONOO⁻ generation. These data show that sufficient supplementation of L-arginine coupled with eNOS can partially restore normal activity of eNOS and bioavailability of NO, therefore the synthesis pathway of ONOO⁻ is turned down at the presence of L-arginine, leading to the reduction of ONOO⁻ production.

Sepiapterin is a precursor of constitutive nitric oxide synthase (cNOS) cofactor tetrahydrobiopterin (BH₄), which can convert to BH₄ via salvage pathway by sepiapterin reductase and dihydrofolate reductase, thereby it can help endothelial NOS maintain functional status with catalytic activity and normal balance between NO and ONOO⁻ by increasing NO biosynthesis from L-arginine.

These data show that uncoupling of NOS stimulated by LDL subclasses can be inhibited or reversed by supplementation of sepiapterin. By restoring the catalytic function of NOS, endothelial cells in sepiapterin treatment group released higher level of NO and lower level of ONOO⁻ than control group after direct injection of LDL with subclasses A, I and B. However, among all of LDL subclasses' injections, subclass A still stimulated the highest level of NO and the lowest level of ONOO⁻, and on the contrary, subclass B stimulated the lowest level of NO and the highest level of ONOO⁻, showing that subclass B is more severe than subclass A in the induction of NOS dysfunction and imbalance of the [NO]/[ONOO⁻] ratio.

As an L-arginine analogue and nonspecific inhibitor of cNOS, L-NAME can bind to the active site of cNOS to block its catalytic activity, resulting in reducing the production of both NO and ONOO⁻. However, this substrate analogue-mediated inhibition of NOS activity is reversible with sufficient supplementation of L-arginine.

Increased NOS activity in low dose treatment of L-NAME may upregulate NO production via feedback regulatory mechanisms, as well as increase the expression level of NOS. However, it does not mean that higher bioavailability of NO is necessarily in association with increased NOS expression and/or NOS activity. Due to the fact that NO biosynthesis is determined by many factors, for instance, the lack of cofactors needed for NOS activation, oxidation and/or inactivation of BH₄ and the presence of highly reactive ROS can reduce NO production.

As the major product of NADPH oxidases and reactive oxygen species (ROS), O₂⁻ can oxidize NO to form ONOO⁻, which contributes to bring oxidative stress to endothelium and lead to endothelial dysfunction. VAS2870 not only permeates cell membrane and inhibits NADPH oxidase activity in a rapid and reversible way, but also repeals agonist-stimulated ROS production and thereby provides protection against oxidative stress generated by ROS.

In these data, NO concentration was increased by 15-24% of control group, showing that this portion of NO produced by endothelial cells is consumed by O₂⁻ generated by nicotinamide adenine dinucleotide phosphate (NADPH) oxidase to form ONOO⁻. Meanwhile, ONOO⁻ concentration was decreased by 20-27% of control group, which was consistent with the increase of NO production (FIG. 5A and FIG. 5B).

Among different subclasses of LDL, subclass B is the most susceptible to be oxidized. Incubation with ox-LDL/n-LDL can stimulate ONOO⁻ release and inhibit NO production from endothelial cells. However, the real-time effect of ox-LDL with different subclasses during direct injection to endothelial cells remains unclear. These data show that injection with ox-LDL stimulated less of NO production and more of ONOO⁻ release than n-LDL, showing that ox-LDL is more cytotoxic than n-LDL in induction of endothelial dysfunction and imbalance of the [NO]/[ONOO⁻] ratio, which may play an important role in the pathogenesis of atherosclerosis.

These data also show the effect of LDL with different subclasses on inducing ICAM-1/VCAM-1 expression and monocyte adhesion to endothelial cells. The data show that LDL significantly up-regulated the expression level of ICAM-1 and VCAM-1, leading to enhancement of monocyte adhesion to endothelial cells. The data also show that monocyte adhesion was positive correlated with the concentration of LDL. Subclass B stimulated the highest level of monocyte adhesion while subclass A stimulated the lowest level of adhesion at same concentration of LDL incubation. Compared with n-LDL treatment group, ox-LDL can stimulate higher level of ICAM-1 and VCAM-1 expression, showing that ox-LDL is more likely to cause monocyte adhesion on the surface of endothelial cells. The data from monocyte adhesion is consistent with the result of ICAM-1 and VCAM-1 expression.

These data also show that n-LDL/ox-LDL with different density can differently alter NO and ONOO⁻ production, and the effect is dose-dependent. The decrease in in cytoprotective NO and the increase of cytotoxic ONOO⁻ shows that subclass B uncouples eNOS bioactivity more significantly than subclasses I and A, causing severe dysfunction in endothelial cells. In addition, subclass B not only stimulated higher expression of ICAM-1 and VCAM-1 than subclasses I and A, but also stimulated maximal monocyte adhesion. It is now believed that subclass B can cause more serious damage to endothelial cells than subclasses A and I, and the distribution of those three LDL subclasses in human blood plays an important role in pathology of cardiovascular diseases.

It is also now believed that elevated levels of subclass B is a leading contributor and/or component of bad cholesterol. Thus, the content of subclass B, in the total bad cholesterol, is useful as a diagnostic predictor in estimating the degree of endothelial dysfunction and cardiovascular system efficiency, and thus further provides a useful diagnostic method in estimating the risk of many cardiovascular diseases.

### Examples

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

Methods

Cell Culture

Human umbilical vein endothelial cells (HUVECs) and human monocytoid cells (THP-1) were purchased from American Type Culture Collection. HUVECs were cultured as monolayer in MCDB-131 Complete Medium (VEC tech) at 37° C. in a humidified atmosphere enriched with 5% CO₂. The THP-1 cells were cultured in RPMI-1640 medium containing 10% FBS (ATCC), 100 U/mL penicillin and 100 U/mL streptomycin at 37° C. in a humidified atmosphere enriched with 5% CO₂.

LDL Isolation, Oxidation and Analysis

Normal human plasma (Innovative Research) was mixed with 12% of OptiPrep density gradient medium (Sigma) at the ratio (v: v) of 1 to 1. The mixture was loaded to the centrifuge tube and placed in NVT65 rotor (Beckman Coulter), then centrifuged at 60,000 rpm (342,000 g) for 4 hours at 16° C. in Optima L-90K ultracentrifuge (Beckman Coulter) set at slow acceleration and slow deceleration. Samples were fractionated within 1 hour after centrifugation. Fractions were collected from each gradient by downward displacement using a syringe tip piercing the bottom of the tube and pumped out. The fractions were collected into Eppendorf tubes with 1.5 ml per fraction. The density and concentration of each fraction were measured by using a refractometer (ATAGO) and cholesterol assay kit (Invitrogen) respectively. Oxidized-LDL (ox-LDL) was prepared. CuSO₄ was added to native LDL (n-LDL) with final concentration of 10 μmol/L. Oxidation was carried out at room temperature over 24 hours until oxidation was complete. The ox-LDL was then placed in ultra-centrifuge tubes (Sigma-Aldrich, Ultra-4, MWCO 30 kDa) and centrifuged at 3000 rpm for 20 minutes to remove CuSO₄. All of the LDL samples were filtered and stored at 4° C.

Nanosensors for Measurement of NO and ONOO⁻

Concurrent measurements of NO and ONOO⁻ were performed with electrochemical nanosensors (diameter: 200-300 nm). The designs of nanosensors are based on chemically modified carbon-fiber technology. Each of those sensors was made by depositing a sensing material on the tip of the carbon fiber. A conductive film of polymeric nickel (II) tetrakis (3-methoxy-4hydroxy-phenyl) porphyrinic was used for the NO sensor and a polymeric film of Mn (III)-paracyclophanyl-porphyrin was used for the ONOO⁻ sensor. NO and ONOO⁻ release from its basal level were measured by using amperometry with time (detection limit of 1 nmol/L and resolution time <50 ms). Each sensor was calibrated by using linear calibration curves from 50 nmol/L to 1000 nmol/L and/or standard addition methods before and after measurements with aliquots of NO or ONOO⁻ standard solutions, respectively.

Determination of n-LDL/Ox-LDL Stimulated NO and ONOO⁻ Production in Endothelial Cells

Endothelial cells were seeded to 24 well plates and cultured in complete medium until confluent monolayer formed. Then the study was carried out as follows:

(a) endothelial cells were stimulated with direct injection of n-LDL with different densities (subclass A: 1.016-1.019 g/mL, subclass I: 1.024-1.029 g/mL, and subclass B: 1.034-1.053 g/mL) and different concentration (50, 100, 250, 500, 750, 1000 μg/mL), and the release of NO/ONOO⁻ was measured by placing a NO/ONOO⁻ nanosensors at a close proximity (5±2 μm; with the help of a micromanipulator, from the surface of endothelial cells and measuring the electrical current generated by NO/ONOO⁻ nanosensors.

(b) Endothelial cells were also stimulated with direct injection of n-LDL with different combinations of subclasses A, B and I LDL (800 μg/mL) as following: (1) 60% A, 20% B and 20% I; (2) 20% A, 60% B and 20% I; (3) 20% A, 20% B and 60% I; (4) 50% A and 50% B; (5) 50% A and 50% I; (6) 50% B and 50% I; (7) 33% A, 38% B and 29% I (simulation of original constituent from general human plasma), and the release of NO/ONOO⁻ was also measured with nanosensors.

(c) Endothelial cells were pre-treated with superoxide dismutase covalently linked to polyethylene glycol (PEG-SOD, 400 U/mL, Sigma), L-arginine (300 μmol/L, Sigma), a precursor of endothelial nitric oxide synthase (eNOS) cofactor tetrahydrobiopterin (sepiapterin, 200 μmol/L, Sigma), L-N^{G}-arginine methyl ester (L-NAME, 100 μmol/L, Sigma) as an inhibitor of eNOS, and a selective nicotinamide adenine dinucleotide phosphate (NADPH) oxidase inhibitor (VAS2870, 10 μmol/L, Sigma) in endothelial basal medium (EBM) at 37° C. for 30 minutes. A control group was incubated in EBM only. After incubation, endothelial cells were stimulated with direct injection of subclasses A, B and I (800 μg/mL), and the release of NO/ONOO⁻ was measured with nanosensors.

(d) Endothelial cells were stimulated with direct injection of n-LDL/ox-LDL (800 μg/mL) and the release of NO/ONOO⁻ was measured in the same way as described above. In separate experiments, the maximal NO and ONOO⁻ concentrations which could be produced by HUVECs was measured after stimulation with 1.0 μmol/L calcium ionophore (A23187, Sigma).

Measurement of Monocyte Adhesion to HUVECs

Endothelial cells were seeded in 96 well plates with complete medium until confluent monolayer formed. THP-1 cells were cultured in RPMI medium 1640 containing 10% FBS, 100 U/mL penicillin, and 100 μg/mL streptomycin at 37° C. in a humidified atmosphere of 5% CO₂. THP-1 cells were pre-labeled with 2′, 7′-bis-(2-carboxyethyl)-5-(and-6)-carboxyl-fluorescein acetoxymethyl ester (BCECF-AM) (Molecular Probes, Life technology) for quantitative adhesion assay. Fluorescence labeling of THP-1 cells was done by incubating cells (5×10⁶ cells/mL) with 5 mol/L BCECF-AM in RPMI-1640 medium for 30 minutes at 37° C. and 5% CO₂. After incubation, cells were washed three times with PBS to remove excess dye. Cells were then re-suspended in EBM at a density of 10⁶ cells/mL. Then the study was carried out as follows:

(1) Confluent HUVECs were incubated with constant concentration (400 μg/mL) of n-LDL at 37° C. for 5 hours. Then cells were washed with PBS twice to remove LDL. Fluorescently labeled THP-1 cells were added to the surface of confluent endothelial monolayer as 10⁵/well and co-incubated at different time intervals (from 10 to 60 minutes); and then the co-cultured cells were washed twice with PBS in order to eliminate the non-adherent cells. The fluorescence intensity of each well was measured by using a fluorescence multi-well plate reader set at excitation and emission wavelengths of 485 and 528 nmol/L, respectively.

(2) Confluent endothelial cells were incubated with LDL at a final concentration of 50, 100, 200 or 400 μg/mL at 37° C. for 5 hours. Then cells were washed with PBS twice to remove LDL. Fluorescently labeled THP-1 cells were added to the surface of confluent endothelial monolayer as 10⁵/well and co-incubated at 37° C. for 1 hour, then the co-cultured cells were washed twice with PBS in order to eliminate the non-adherent cells. The fluorescence intensity of each well was measured in the same way as described above.

(3) Confluent endothelial cells were incubated with n-LDL/ox-LDL (400 μg/mL) at 37° C. for 5 hours. After that, cells were washed with PBS twice to remove LDL and fluorescently labeled THP-1 cells were added to confluent endothelial monolayer (10⁵/well) and co-incubated at 37° C. for 1 hour. The co-cultured cells were then washed twice with PBS in order to eliminate the non-adherent cells. The fluorescence intensity of each well was measured in the same way as described above.

Measurements of Adhesion Molecules

Cell ELISA was used to measure the expression of adhesion molecules. Endothelial cells were seeded in 96 well plates with complete medium until confluent monolayer formed. Then cells were incubated with n-LDL/ox-LDL (400 μg/mL) at 37° C. for 5 hours. Control is EBM with 3% iodixanol. After stimulation with LDL, endothelial cells were washed with phosphate buffered saline (PBS) twice and fixed with 4% formaldehyde solution for 20 minutes at room temperature. After fixation, HUVECs were washed twice with phosphate buffered saline with tween 20 (PBST) and incubated with blocking buffer (4% BSA in PBST) for 1 hour at room temperature. The plate was washed three times with PBST and primary monoclonal antibody against ICAM-1 and VCAM-1 (Santa Cruz) diluted in PBST (0.5 μg/mL for ICAM-1, and 2 μg/mL for VCAM-1) were added to the cells at 4° C. overnight. The plate was washed three times with PBST and incubated with horseradish peroxidase-conjugated goat anti-mouse IgG (Santa Cruz) diluted at 1:1000 in PBST for 1 hour at room temperature. The cells were washed again three times, and 4,4′-Bi-2,6-xylidine; 4,4′-diamino-3,3′,5,5′-tetramethylbiphenyl (TMB) solution was added to each well and incubated at room temperature. After then, 2M citric acid solution was added to each well. The absorbance was measured at 450 nm wavelength in a microplate reader. Each experiment was performed in six duplicates and repeated at least three times.

Statistical Analysis

All data are expressed as means±SD. Unpaired Student's t-test was used to measure statistical differences. A P value less than 0.01 was considered statistically significant. Data analysis was performed using Excel version 2013 (Microsoft, Seattle, Wash.). Asterisk in the figures represents as following: *: P<0.01.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

1. A method of determining potential risk to a subject's cardiovascular system, comprising:
measuring, in situ, the concentrations of nitric oxide [NO] and peroxynitrite [ONOO⁻] stimulated by the different subclasses of (LDL) in one or more cells of the subject;
wherein a concentration ratio of [NO]/[ONOO⁻] below 1.0 indicates an imbalance between cytoprotective NO and cytotoxic ONOO⁻.

2. The method of claim 1, using an assay to measure the relative concentration of the subclasses (A, I and B) of LDL and their reaction with endothelial cells.

3. The method of claim 1, wherein the method comprises diagnosing general cardiac risk factors based on the measurement of subclass B of LDL, comprising:
measuring the concentration of LDL-B by ultracentrifugation or electrophoresis methods; and,
comparing the measured concentrations to determine the concentration of LDL-B as compared to the total concentration of LDL;
wherein a score in % is then compared with a calibration curve, based on nanomedical measurements of NO and ONOO− concentrations, which are produced by a model of endothelial cells (mixed population); and,
wherein a score of 40%, or higher, on this scale indicates increased risk of cardiovascular diseases (CVD).

4. The method of claim 1, wherein the method comprises diagnosing cardiovascular risk are based on the simultaneous measurements of all the subclasses of LDL (A, I and B), comprising:
i) separating three subclasses from a sample, and quantitatively measured using either electrophoresis, ultracentrifugation, or immunoseparation methods;
ii) comparing the concentration of each LDL subclass, separately; thereafter, and
iii) comparing the concentration of LDL-B to the sum of LDL-1 and LDL-A concentrations;
iv) constructing a calibration curve from the calibration data from NO and ONOO− concentrations produced by endothelial cells after stimulation with different combinations of LDL-A, LDL-B and LDL-I.

5. The method of claim 1, wherein the method comprises personalized diagnosis of a patient to estimate the risk for cardiovascular disease (CVD), endothelial dysfunction, and/or the rate and time of the progression of vascular disease comprising:
i) harvesting endothelial cells and/or platelets from the patient;
ii) stimulating NO and ONOO− concentrations generated by the endothelial cells and/or platelets with each subclass of LDL, both separately and in combination; AB, AI and BI by:
a) separating three subclasses from a sample, and quantitatively measured using either electrophoresis, ultracentrifugation, or immunoseparation methods;
b) comparing the amount/concentration of each LDL subclass, separately; thereafter,
c) comparing the amount/concentration of LDL-B to the sum of LDL-1 and LDL-A concentrations; and,
d) constructing a calibration curve from the calibration data from NO and ONOO⁻ concentrations produced by endothelial cells after stimulation with different combinations of LDL-A, LDL-B and LDL-1;
iii) determining a personal diagnosis based to accurately estimate the risk for CVD, as well as the level of endothelial dysfunction, the rate and time of the progression of vascular disease; and,
iv) administering a suitable pharmacological treatment, optionally, using selective LDL-B statins, L-arginine, vitamin D3 and others.

6. (canceled)

7. The method of claim 1, wherein the method comprises of determining potential risk to a subject's cardiovascular system, comprising:
i) obtaining a sample from the subject having at least one cell having low density lipoproteins (LDLs) therein,
wherein the LDLs are comprised of the subclasses of LDL with distinct densities:
n-LDL subclass A having a density of 1.025-1.034 g/mL;
n-LDL subclass I having a density of 1.034-1.044 g/mL; and,
n-LDL subclass B having a density of 1.044-1.060 g/mL;
ii) measuring concentration of NO and ONOO⁻ released from the cell;
iii) determining the ratio of cytoprotective NO concentration to cytotoxic ONOO⁻ concentration [NO]/[ONOO⁻];
wherein a balance of [NO]/[ONOO⁻] in normal endothelium is about 5, but is shifted to 2.7±0.4, 0.5±0.1 and 0.9±0.1 for n-LDL subclasses A, B and I, respectively;
wherein a ratio below 1.0 indicates an imbalance between cytoprotective NO and cytotoxic ONOO⁻, which negatively affects endothelial function.

8. The method of claim 7, wherein a high content/level of subclass B LDL in total cholesterol is a determinant of potential risk for the subject's cardiovascular system.

9. An assay for diagnosing whether a subject has cardiovascular disease (CVD), comprising:
i) obtaining, or having obtained, at least one cell from the subject;
ii) exposing the cell to at least one nanosensor to measure the concentration NO and ONOO⁻ released from the cell; and,
iii) determining a ratio of cytoprotective NO concentration to cytotoxic ONOO⁻ concentration [NO]/[ONOO⁻];
wherein a ratio of [NO]/[ONOO⁻] in normal endothelium is about 5, but is shifted to 2.7±0.4, 0.5±0.1 and 0.9±0.1 for native LDL (n-LDL) subclasses A, B and I, respectively; and,
wherein a ratio below 1.0 indicates an imbalance between cytoprotective NO and cytotoxic ONOO⁻.

10. The assay of claim 9, wherein a high content/level of subclass B LDL in total cholesterol is a determinant of potential risk for the cardiovascular system.

11. The assay of claim 9, wherein the nanosensor comprises a chemically modified carbon-fiber.

12. The assay of claim 9, wherein the nanosensor comprises a NO sensing material and an ONOO⁻ sensing material deposited on the tip of a carbon fiber.

13. The assay of claim 9, wherein the NO sensing material comprises a conductive film of polymeric nickel (II) tetrakis (3-methoxy-4hydroxy-phenyl) porphyrinic; and/or wherein the ONOO⁻ sensing material comprises a polymeric film of Mn (III)-paracyclophanyl-porphyrin.

14. The assay of claim 9, wherein the NO and ONOO⁻ released are measured by using amperometry with time (detection limit of 1 nmol/L and resolution time <50 ms).

15. The assay of claim 9, wherein the nanosensor is calibrated by using linear calibration curves from 50 nmol/L to 1000 nmol/L and/or standard addition methods before and after measurements with aliquots of NO or ONOO⁻ standard solutions, respectively.

16. A method for diagnosing whether a subject has cardiovascular disease (CVD), comprising:
determining CVD progression in the subject by measuring the increased ratio of subclass B LDL in a sample from the subject, as compared to subclasses A and I and/or a previous measured sample from the subject.

17. The method of claim 16, wherein a balance of [NO]/[ONOO⁻] in normal endothelium is about 5, but is shifted to 2.7±0.4, 0.5±0.1 and 0.9±0.1 for native LDL (n-LDL) subclasses A, B and I, respectively; and, wherein a ratio below 1.0 indicates an imbalance between cytoprotective NO and cytotoxic ONOO⁻.

18. The method of claim 16, wherein the method is used for mass screening of patients.

19. The method of claim 16, wherein the method further comprises: determining the phase of CVD in the subject by distinguishing among ratios of subclasses A, I and B of LDL.

20. The method of claim 6, further comprising:
i) obtaining, or having obtained, a sample of the at least one cell from the subject;
ii) contacting the sample with a nanosensor; and,
iii) measuring the concentration of NO and ONNO⁻ in the sample,
wherein a ratio of [NO]/[ONOO⁻] in normal endothelium is about 5, but is shifted to 2.7±0.4, 0.5±0.1 and 0.9±0.1 for native LDL (n-LDL) subclasses A, B and I, respectively.

21. A method of treating cardiovascular disease (CVD) in a subject in need thereof, comprising:
i) conducting the assay of claim 9, and
ii) treating the subject if the assay shows the presence of changes in the ratios of at least one of the subclasses A, I and B of LDL.

22. The method of claim 16, further comprising reducing the risk of cardiovascular disease (CVD) progression or reducing the risk of recurrence of a CVD in remission in a subject, by;
i) screening for changes in the ratios of at least one of the subclasses A, I and B of LDL; and,
ii) treating the subject if the screening shows the presence of changes in the ratios of at least one of the subclasses A, I and B of LDL.

23. An in vitro method for determining a drug-responding or non-responding phenotype in a subject suffering from a cardiovascular disease (CVD), comprising the steps of:
i) determining from a biological sample of a subject, the ratios of the (LDL subclasses, including measuring subclass B to A+I or B to A;
ii) comparing the level in step a) to a reference level; and,
iii) determining the drug-responding or non-responding phenotype from said comparison.

24. A method for designing or adapting a treatment regimen for a subject suffering from cardiovascular disease (CVD), comprising the steps of:
i) determining from a biological sample of said subject a drug-responding or non-responding phenotype according to the method of claim 23; and,
ii) designing or adapting a treatment regimen for said subject based upon said responding or non-responding phenotype.

25. A method for diagnosing cardiovascular disease (CVD) in a subject, the method comprising:
i) obtaining, or having obtained, a sample from a subject suspected of having CVD;
ii) contacting the sample with one or more nanosensors;
iii) detecting and/or quantifying ratios of the subclasses B to A+I, or the B to A ratio of LDL present in the patient sample; thereby obtaining a patient sample subclass-LDL value;
iv) comparing the patient sample value to a reference value; wherein the reference value is set based on one or more individuals that do not have CVD; and,
v) diagnosing CVD if the patient sample value is below the reference value.

26. The method of claim 25, wherein the reference value is set by:
a) obtaining one or more normal samples from one or more individuals that do not have cardiovascular disease (CVD);
b) contacting the one or more normal samples with a nanosensor;
c) detecting and/or quantifying ratios of at least one of the subclasses A, I and B of LDL present in the one or more normal samples; thereby obtaining one or more reference values; and,
d) setting the reference value based on the one or more normal sample values.

27. The method of claim 26, further comprising:
e) applying the measured value from the subject against a database of measured values from control subjects, wherein the database is stored on a computer system; and,
f) determining that the subject has an increased risk of having CVD or disorder by measuring a change of at least 5% in the value relative to measured value from control subjects.

28. The method of claim 27, wherein a subject has a risk of having or has CVD if the measurements show a change in expression of at 10% compared to a control subject population.

29. The method of claim 27, wherein the subject who has a change in expression of at least 10% compared to a control subject population, is further screened for CVD.

30. The method of claim 27, where sample comprises one or more of: blood or endothelial cells.

31. A computer-based method for screening a subject for the presence of and treating cardiovascular disease (CVD), comprising: screening the subject by:
i) obtaining, or having obtained, a sample comprising at least one cell from the subject,
ii) exposing the sample to at least one nanosensor to measure the concentration of NO and ONOO⁻ released from the cell, and,
iii) determining the ratio of cytoprotective NO concentration to cytotoxic ONOO⁻ concentration [NO]/[ONOO⁻];
wherein a balance of [NO]/[ONOO⁻] in normal endothelium is about 5, but is shifted to 2.7±0.4, 0.5±0.1 and 0.9±0.1 for native LDL (n-LDL) subclasses A, B and I, respectively; and,
wherein a ratio below 1.0 indicates an imbalance between cytoprotective NO and cytotoxic ONOO⁻;
iv) applying a measured ratio value from the subject against a database of measured ratio values from control subjects, wherein the database is stored on a computer system;
v) determining that the subject has an increased risk of having CVD by measuring a change of at least 5% in the subject's ratio value relative to measured ratio values from control subjects;
vi) performing a diagnostic procedure comprising downloading the plurality of subject's ratio values into a computer processor; and
vii) administering an effective anti-CVD treatment to the subject.

32. The method of claim 31, wherein the anti-CVD treatment comprises administering an effective amount of a pharmaceutical composition to restore the catalytic function of NOS in the subject's cells.

33. The method of claim 31, wherein the pharmaceutical compositions are selected from one or more: combinations of vitamin D₃, L-arginine, apocynin, and statins with selected capability to restore damage imposed by subclass B of LDL on NOS function.

34. A kit intended for the detection of cardiovascular disease (CVD) in a sample, the kit comprising: at least one nanosensor, and instructions for obtaining a ratio value of cytoprotective NO concentration to cytotoxic ONOO⁻ concentration [NO]/[ONOO⁻].

35. The kit of claim 34, wherein a balance of [NO]/[ONOO⁻] in normal endothelium is about 5, but is shifted to 2.7±0.4, 0.5±0.1 and 0.9±0.1 for n-LDL subclasses A, B and I, respectively; and, wherein a ratio below 1.0 indicates an imbalance between cytoprotective NO and cytotoxic ONOO⁻, which affects endothelial function.

36. The kit of claim 34, wherein the nanosensor comprises a chemically modified carbon-fibers.

37. The kit of claim 34, wherein the nanosensor comprises a NO sensing material and an ONOO⁻ sensing material deposited on the tip of a carbon fiber.

38. The kit of claim 34, wherein the NO sensing material comprises a conductive film of polymeric nickel (II) tetrakis (3-methoxy-4hydroxy-phenyl) porphyrinic; and/or wherein the ONOO⁻ sensing material comprises a polymeric film of Mn (III)-paracyclophanyl-porphyrin.

39. The kit of claim 34, wherein NO and ONOO⁻ released are measured by using amperometry with time (detection limit of 1 nmol/L and resolution time <50 ms).

40. The kit of claim 34, wherein the nanosensor is calibrated by using linear calibration curves from 50 nmol/L to 1000 nmol/L and/or standard addition methods before and after measurements with aliquots of NO or ONOO⁻ standard solutions, respectively.
